(19) United States
(12) Patent Application Publication
(10) Pub. No.: US 2025/0339659 A1
(43) Pub. Date: Nov. 6, 2025

(54) MICRONEEDLE STRUCTURE AND METHOD FOR PRODUCING MICRONEEDLE STRUCTURE

(71) Applicant: LINTEC CORPORATION, Tokyo (JP)

(72) Inventors: Yosuke KOMA, Tokyo (JP); Akio KABUTO, Tokyo (JP)

(21) Appl. No.: 18/851,452

(22) PCT Filed: Mar. 30, 2023
(86) PCT No.: PCT/JP2023/013269

§ 371 Date: Sep. 26, 2024

(30) Foreign Application Priority Data
Mar. 31, 2022 (JP) 2022-059926
Jun. 30, 2022 (JP) 2022-106672
Jun. 30, 2022 (JP) 2022-106673

(51) Int. Cl. A61M 37/00

(52) U.S. Cl. CPC A61M 37/0015; A61M 2037/0023; A61M 2037/0053

(57) ABSTRACT

Microneedle structure includes needle-shaped portions having hole portions formed therein and a base material having one surface side on which the needle-shaped portions are formed. Each of the needle-shaped portions is formed with a porous structure, and the value of tip strength of the needle-shaped portions is 40 mN or more. The method for producing a microneedle structure is used to produce the microneedle structure. Thus, it is possible to provide: a microneedle structure having needle-shaped portions that are suppressed in the defect or breakage during use; and a method for producing such a microneedle structure.

### TECHNICAL FIELD

The present invention relates to a microneedle structure and a method for producing a microneedle structure.

### BACKGROUND ART

In recent years, there have been proposals for supplying a drug into the body or collecting body fluids from the body through through-holes formed in microneedles. For example, microneedles are known, which include a microneedle-shaped biocompatible matrix and porous particles provided on or at least partially in the biocompatible matrix (Patent Document 1).

### PRIOR ART DOCUMENTS

### Patent Documents

[Patent Document 1] JP2014-094171A

### SUMMARY OF THE INVENTION

### Problems to be Solved by the Invention

Patent Document 1 assumes that the microneedles may be absorbed in the body because biocompatible materials constituting the microneedles will swell or will be absorbed into biological tissue within a few seconds to a few hours when they are pierced into the skin. From the viewpoint of safety, however, it is desirable to remove the pierced microneedles so as not to leave them in the skin as much as possible. Here, if microneedles containing porous particles as disclosed, for example, in Patent Document 1 are pierced and then removed from the skin, there is a problem in that the microneedles may become defective. In addition, there is a possibility that the efficiency of supplying a drug or the like may be reduced due to breakage when the microneedles are pierced into the skin.

The present invention has been made in view of such actual circumstances, and an object of the present invention is to provide a microneedle structure having a needle-shaped portion that is suppressed in its defect or breakage during use. Another object of the present invention is to provide a method for producing such a microneedle structure.

### Means for Solving the Problems

To achieve the above objects, first, the present invention provides a microneedle structure comprising: a needle-shaped portion having an interior formed with a hole portion; and a base material having one surface side on which the needle-shaped portion is formed, the needle-shaped portion being formed with a porous structure, the needle-shaped portion having a value of tip strength of 40 mN or more as measured by an evaluation method below, the evaluation method comprising: placing the microneedle structure on a stage with the needle-shaped portion facing up; observing the microneedle structure with a microscope to select one needle-shaped body having a sharp tip shape; aligning an attachment (made of iron, 2 mmφ) of a digital force gauge with a position of the needle-shaped portion of the needle-shaped body; lowering the attachment at a speed of 5 mm/min to measure a force applied to the attachment (measurement environment temperature: 23° C., measurement environment relative humidity: 50%); at a point of time when a decrease in the force is first observed on a graph on which the measured force is output, reading a local maximum value of the force exhibited at a position before the decrease in the force, or reading a value of the force at a point of time when a fall of the attachment reaches 100 μm if the decrease in the force is not observed until the fall of the attachment reaches 100 μm; and determining the read value as the tip strength of the needle-shaped portion (Invention 1).

In the above invention (Invention 1), the value of the tip strength of the needle-shaped portion is 40 mN or more, so that it is possible to suppress the breakage of the needle-shaped portion when it is pierced into the skin.

In the above invention (Invention 1), the hole portion may be preferably opened on a side surface of the needle-shaped portion (Invention 2).

In the above invention (Invention 1), preferably, the needle-shaped portion may contain a high-melting-point resin whose melting point exceeds 130° C., the base material may contain a layer containing a heat-resistant resin, and a liquid can pass through the base material in its thickness direction (Invention 3).

In the above invention (Invention 3), the high-melting-point resin may be preferably a water-insoluble resin (Invention 4).

In the above invention (Invention 3), the high-melting-point resin may be preferably a biodegradable resin (Invention 5).

In the above invention (Invention 3), the high-melting-point resin may be preferably a copolymer of at least one monomer selected from polylactic acid and polyglycolic acid and another monomer (Invention 6).

In the above invention (Invention 3), the layer containing a heat-resistant resin may preferably contain at least one heat-resistant organic polymer selected from polymethylmethacrylate, polystyrene, polyacrylonitrile, polyphenylene oxide, polyethylene naphthalate, polyphenylene sulfide, polytetrafluoroethylene, polycarbonate, allyl resin, polyether ether ketone, acetyl cellulose resin, polysulfone, polyether sulfone, polyimide, and polyamide imide, a copolymer obtained by copolymerizing a monomer that is a raw material of the heat-resistant organic polymer and any other monomer, or a silicone resin (Invention 7).

In the above invention (Invention 3), the base material and the needle-shaped portion may be preferably directly bonded via a base portion formed of a same material as that of the needle-shaped portion (Invention 8).

In the above invention (Invention 1), the needle-shaped portion may preferably contain a low-melting-point resin whose melting point is 130° C. or lower (Invention 9).

In the above invention (Invention 1), the needle-shaped portion may preferably contain a high-melting-point resin and a low-melting-point resin whose melting point is 130° C. or lower (Invention 10).

Second, the present invention provides a method for producing a microneedle structure comprising: a needle-shaped portion having an interior formed with a hole portion; and a base material having one surface side on which the needle-shaped portion is formed, the method comprising a formation step of heating a composition containing a high-melting-point resin whose melting point exceeds 130° C. to form a projecting portion on the base material by the composition (Invention 11).

In the above invention (Invention 11), preferably, the high-melting-point resin may be a water-insoluble resin, the composition may be a mixture of the water-insoluble resin and a water-soluble material, and the method may comprise a removal step of, after the formation step, removing with a water-containing solution the water-soluble material of the formed projecting portion to form a hole portion in the projecting portion (Invention 12).

Third, the present invention provides a method for producing a microneedle structure comprising: a needle-shaped portion having an interior formed with a hole portion; and a base material having one surface side on which the needle-shaped portion is formed, the method comprising a bonding step of heating a composition containing a high-melting-point resin whose melting point exceeds 130° C. to bond the heated composition and the base material (Invention 13).

### BRIEF DESCRIPTION OF DRAWINGS

FIG. 1 is a set of (1) a schematic cross-sectional diagram of a microneedle structure of the present invention and (2) a partial enlarged view of a needle-shaped portion.

FIG. 2 is a schematic partial cross-sectional diagram of a test patch using the microneedle structure of the present invention.

FIG. 3 is a set of explanatory diagrams (a) to (c) illustrating the procedure of a method for producing a microneedle structure according to an embodiment.

FIG. 4 is a set of explanatory diagrams (a) to (c) illustrating the procedure of a method for producing a microneedle structure according to an embodiment.

### EMBODIMENTS FOR CARRYING OUT THE INVENTION

Hereinafter, embodiments of the present invention will be described.

### <Microneedle Structure>

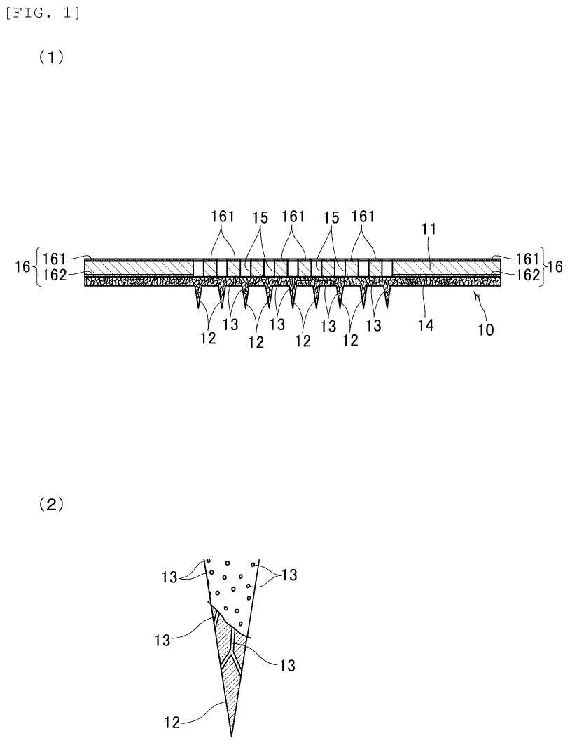

FIG. 1 illustrates a microneedle structure 10 according to an embodiment of the present invention. The microneedle structure 10 includes a plurality of needle-shaped portions 12 that are spaced apart from each other at predetermined intervals on one surface side of a base material 11. The base material 11 is formed with through-holes 15. In addition, the needle-shaped portions 12 are each formed with a plurality of hole portions 13. The microneedle structure 10 can be used as a test patch that absorbs a body fluid from inside the skin through the hole portions 13 of the needle-shaped portions 12 and performs a test using the body fluid obtained via the base material 11 or can also be used as a drug administration patch that administers a drug from the skin into the body via the base material 11 and the hole portions 13 of the needle-shaped portions 12. In the present invention, the body fluid refers blood, lymph, interstitial fluid, etc.

### (1) Needle-Shaped Portions

The shape, size, formation pitch, and number of formation of the needle-shaped portions 12 can be appropriately selected depending on the intended use of the microneedles. Examples of the shape of the needle-shaped portions 12 include columnar, prismatic, conical, and pyramidal shapes. In the present embodiment, the shape of the needle-shaped portions 12 is pyramidal. The maximum diameter or maximum cross-sectional dimension of the needle-shaped portions 12 may be, for example, 25 to 1,000 μm. The tip diameter or cross-sectional dimension of tips may be 1 to 100 μm. The height of the needle-shaped portions 12 may be, for example, 50 to 2,000 μm. The needle-shaped portions 12 may be arranged in a plurality of rows in one direction of the base material 11, and each row may be provided with a plurality of needle-shaped portions 12 to form a matrix.

In the present embodiment, the needle-shaped portions 12 have a value of tip strength of 40 mN or more as measured by the following evaluation method. The valuation method includes: placing the microneedle structure on a stage with the needle-shaped portions facing up; observing the microneedle structure with a microscope to select one needle-shaped body having a sharp tip shape; aligning an attachment (made of iron, 2 mmφ) of a digital force gauge with the position of the needle-shaped portion of the needle-shaped body; lowering the attachment at a speed of 5 mm/min to measure a force applied to the attachment (measurement environment temperature: 23° C., measurement environment relative humidity: 50%); at a point of time when a decrease in the force is first observed on a graph on which the measured force is output, reading a local maximum value of the force exhibited at a position before the decrease in the force, or reading a value of the force at a point of time when a fall of the attachment reaches 100 μm if the decrease in the force is not observed until the fall of the attachment reaches 100 μm; and determining the read value as the tip strength of the needle-shaped portion.

Details of the tip strength evaluation method include, for example, a method of evaluating the tip strength employing the procedure, device, etc. described in Examples, which will be described later. By setting the tip strength of the needle-shaped portions 12 at such a value, it is possible to suppress the breakage of the needle-shaped portions 12 when piercing them into the skin. From such a viewpoint, the tip strength of the needle-shaped portions may be preferably 40 to 500 mN, more preferably 60 to 450 mN, further preferably 85 to 400 mN, and furthermore preferably 100 to 350 mN.

In the present embodiment, particularly in a first embodiment regarding a resin that constitutes the needle-shaped portions 12 (also simply referred to as “the first embodiment”), the needle-shaped portions 12 may be preferably composed of a high-melting-point resin. The high-melting-point resin may be preferably one having a melting point of higher than 130° C., further preferably one having a melting point of 135° C. to 240° C., more preferably one having a melting point of 140° C. to 220° C., and most preferably one having a melting point of 145° C. to 200° C. High-melting-point resins are less likely to soften at temperatures near an ordinary temperature at which the microneedle structure 10 is used. Therefore, by containing in the needle-shaped portions 12 a high-melting-point resin whose melting point exceeds 130° C., it is possible to maintain the sufficient strength. As will be described later, in the case of a structure in which a plurality of hole portions 13 are opened on the side surface of each needle-shaped portion 12, it is possible to increase the rate of absorption or release of fluid from the needle-shaped portions 12 as compared with a structure in which hole portions are opened only at the top of each needle-shaped portion, but the needle-shaped portions 12 may become brittle and the strength tends to decrease. Fortunately, however, in the present embodiment, since the needle-shaped portions 12 are formed of a composition that contains a high-melting-point resin whose melting point exceeds 130° C., the strength can be increased, and it is possible to suppress the breakage of the needle-shaped portions 12, for example, when piercing them into the skin.

Such a high-melting-point resin may be preferably a water-insoluble high-melting-point resin. By being water-insoluble, the resin is not dissolved with body fluids when applied to a living body, and it is possible to maintain the shape of the microneedle structure 10 for a desired application time. Moreover, as will be described later, the fine hole portions 13 can be readily formed. In the present embodiment, the needle-shaped portions 12 may be composed of a water-insoluble material that contains a water-insoluble high-melting-point resin. Examples of water-insoluble high-melting-point resins other than the biodegradable resin described later include polypropylene, polyvinylidene fluoride, acetal resin, and polycarbonate.

The molecular weight of the high-melting-point resin may be usually 5,000 to 1,000,000, preferably 7,000 to 500,000, and more preferably 9,000 to 300,000. When the molecular weight is within this range, the needle-shaped portions 12 can be preferably formed.

The high-melting-point resin may be preferably a high-melting-point biodegradable resin. Here, the biodegradable resin is a plastic that is eventually completely decomposed into CO₂ and water after use by the action of microorganisms present in nature. By being a biodegradable resin, it is possible to reduce the influence on a living body. As such a biodegradable resin, aliphatic polyesters and their derivatives may be preferably used. Specific examples thereof include polyglycolic acid (melting point: 218° C.), polylactic acid (melting point: 170° C.), and polyhydroxybutyric acid (melting point: 175° C.). Such examples also include a copolymer composed of two or more monomers selected from the group consisting of glycolic acid, lactic acid, and caprolactone. From the viewpoint of having a melting point exceeding 130° C., such a copolymer may be preferably one containing glycolic acid or lactic acid as the main monomer component. The high-melting-point biodegradable resin may be preferably polyglycolic acid, polylactic acid, or a copolymer of glycolic acid and lactic acid, and polylactic acid may be more preferred.

In the present embodiment, particularly in a second embodiment regarding a resin that constitutes the needle-shaped portions 12 (also simply referred to as “the second embodiment”), the resin that constitutes the needle-shaped portions 12 may be a low-melting-point resin. Materials for the low-melting-point resin may be solid at an ordinary temperature and may preferably have a melting point of 130° C. or lower, more preferably lower than 130° C., particularly preferably 40° C. to 120° C., and most preferably 45° C. to 100° C. When the low-melting-point resin is solid at an ordinary temperature, the needle-shaped portions 12 can maintain their shapes at an ordinary temperature. Provided that the melting point is 130° C. or lower, when the resin is melted for forming the needle-shaped portions 12, high-temperature heating is not necessary, and good workability can be obtained at low cost. In addition, even when the resin is bonded in a molten state to the base material 11 or the resin is heated in a state in which the resin and the base material are bonded, the resin can be melted at a low temperature, so the base material 11 is not softened, deformed, or burned, and the degree of freedom in selecting the base material 11 is high. Furthermore, even when a non-woven fabric, resin film, or the like whose material is a synthetic fiber or the like having a low heat resistance temperature, for example, is used as the base material 11, deterioration of the base material 11 due to softening or the like of the synthetic fiber can be prevented. From such a viewpoint, the ratio of the low-melting-point resin to the total mass of the resin components contained in the needle-shaped portions 12 may be preferably 50 mass % or more and more preferably 70 mass % or more

The low-melting-point resin that constitutes the needle-shaped portions 12 may be a water-insoluble resin. By being water-insoluble, the resin is not dissolved with body fluids when applied to a living body, and it is possible to maintain the shape of the microneedle structure 10 for a desired application time. Moreover, as will be described later, the fine hole portions 13 can be readily formed. Examples of water-insoluble resins include: polyolefin-based resins such as polyethylene and α-olefin copolymers; olefin copolymer-based resins such as ethylene-vinyl acetate copolymer resins; polyurethane-based elastomers; and acrylic copolymer-based resins such as ethylene-ethyl acrylate copolymers.

The low-melting-point resin that constitutes the needle-shaped portions 12 may also be a biodegradable resin. Preferred examples of such biodegradable resins for use include aliphatic polyesters and derivatives thereof, homopolymers of at least one monomer selected from the group consisting of glycolic acid, lactic acid, and caprolactone, and copolymers composed of two or more monomers. In addition, polybutylene succinate (melting point: 84° C. to 115° C.), aliphatic-aromatic copolyester (melting point: 110° C. to 120° C.), etc. can also be used as the low-melting-point biodegradable resin. Specific examples of the polybutylene succinate for use include BioPBS provided by Mitsubishi Chemical Corporation, and specific examples of the aliphatic-aromatic copolyester for use include Ecoflex available from BASF.

The biodegradable resin may be a resin whose monomer acid dissociation constant is 4 or more. When the monomer acid dissociation constant is 4 or more, it is possible to reduce the influence on a living body upon application of the microneedle structure 10 of the present invention to the living body. When the monomer is a cyclic ester, the monomer acid dissociation constant as referred to herein is the acid dissociation constant of the hydroxycarboxylic acid resulting from ring-opening of the cyclic ester. The monomer acid dissociation constant may be preferably 4.0 or larger and further preferably 4.5 or larger. From another aspect, the monomer acid dissociation constant may be preferably 25 or less and further preferably 15 or less. Examples of monomers constituting such a biodegradable resin and having an acid dissociation constant of 4 or more include caprolactone. The constituent units of monomers having an acid dissociation constant of 4 or more from which the low-melting-point biodegradable resin is derived may preferably account for 70 mass % or more, more preferably 80 mass % or more, and further preferably 90 mass % or more in the entire constituent units.

The molecular weight of the resin constituting the needle-shaped portions 12 may be usually 5,000 to 300,000, preferably 7,000 to 200,000, and more preferably 8,000 to 150,000.

Most preferably, the low-melting-point resin constituting the needle-shaped portions 12 may be a water-insoluble low-melting-point resin that is also biodegradable. Examples thereof include polycaprolactone or a copolymer of caprolactone and another polymer, whose monomer acid dissociation constant is 4 or more.

In the second embodiment, the resin constituting the needle-shaped portions 12 may be preferably a low-melting-point resin whose weight-average molecular weight is 40,000 or more, that is, a high-molecular-weight low-melting-point resin.

The weight-average molecular weight of the high-molecular-weight low-melting-point resin may be preferably 40,000 or more, more preferably 40,000 to 200,000, and further preferably 60,000 to 150,000. When the weight-average molecular weight is within this range, the tip strength of the needle-shaped portions 12 can readily be improved.

In the second embodiment, the needle-shaped portions 12 may preferably further contain a filler. By containing a filler in the needle-shaped portions 12, it is possible to further improve the mechanical strength of the needle-shaped portions 12. The filler may be preferably contained so that it is in a dispersed state in the resin of the needle-shaped portions 12.

The filler may be preferably composed of a resin, or one selected from the group consisting of natural organic polymers or modified products thereof and biodegradable resins. The filler for use composed of a resin may be one that contains an inorganic component, etc., such as an organic/inorganic hybrid filler in which an inorganic material is attached to the surfaces of resin particles, but considering the influence on a living body, the filler may be preferably composed only of a resin and an organic component, and more preferably composed only of a resin. Examples of natural organic polymers include cellulose, and examples of fillers composed of natural organic polymers or modified products thereof f include cellulose fibers and cellulose acetate true spherical particles.

The biodegradable resins described above can be used, but when a low-melting-point biodegradable resin is used as the resin constituting the needle-shaped portions 12, it is preferred to use a biodegradable resin different from this biodegradable resin, and from the viewpoint of further improving the mechanical strength of the filler as described below, a biodegradable resin whose melting point exceeds 130° C. or a biodegradable resin with no melting point may be preferred. Examples of such biodegradable resins include polylactic acid (melting point: 170° C.), polyglycolic acid (melting point: 218° C.), polyhydroxybutyric acid (melting point: 175° C.), and cellulose acetate diacetate (melting point: 230° C. to 300° C.). Biodegradable resins such as cellulose acetate diacetate also fall under modified products of natural organic polymers.

From the viewpoint of further improving the mechanical strength of the needle-shaped portions 12, the filler may be preferably composed of a resin whose melting point exceeds 130° C. or a resin having no melting point. Resins whose melting point exceeds 130° C. are less likely to soften at temperatures near an ordinary temperature at which the microneedle structure 10 is used. Therefore, when the filler is composed of a resin whose melting point exceeds 130° C., it is easy to obtain sufficient strength of the microneedle structure 10. In addition, when the filler is composed of a resin whose melting point exceeds 130° C., addition of such a resin in the form of a filler may be preferred because when such a resin that is difficult to melt is mixed with a low-melting-point resin in a state in which the filler is dispersed in the composition, production is possible through low-temperature kneading without melting of the filler. Other than the above-described biodegradable resins, examples of a resin whose melting point exceeds 130° C. and a resin having no melting point include polypropylene (melting point: 155° C.), polybutylene terephthalate (223° C.), polyethylene terephthalate (melting point: 260° C.), polytetrafluoroethylene (melting point: 327° C.), melamine resin (melting point: none), and unmodified cellulose (melting point: none).

From the same viewpoint, the filler may also be preferably composed of a resin whose glass-transition temperature is −10° C. or higher. From another aspect, the filler may be preferably composed of a resin whose glass-transition temperature is 80° C. or lower. Provided that the filler is composed of a resin whose glass-transition temperature is 80° C. or lower, even when melting is performed at a low temperature, the filler is readily softened during the melting and is more compatible with a low-melting-point resin. This can readily improve the strength of the needle-shaped portions 12 to be produced. When the resin contained in the filler is crosslinked, the glass-transition temperature of the polymer being −10° C. or higher or 80° C. or lower is determined before crosslinking. Examples of resins whose glass-transition temperature (Tg) is −10° C. or higher and 80° C. or lower include polypropylene (Tg: 0° C.), polybutylene terephthalate (Tg: 50° C.), polyethylene terephthalate (Tg: 69° C.), polymethyl methacrylate (Tg: 60° C.), polylactic acid (Tg: 60° C.), polyglycolic acid (Tg: 40° C.), and polyhydroxybutyric acid (Tg: 15° C.), among which, as described above, a biodegradable resin may be preferred, and polylactic acid, polyglycolic acid, polyhydroxybutyric acid, or a copolymer of monomers of these polymers may be preferred.

The filler may more preferably composed of a resin whose glass-transition temperature is 10° C. to 80° C., and further preferably composed of a resin whose glass-transition temperature is 30° C. to 75° C.

The filler may be preferably contained in an amount of 3 to 50 mass %, more preferably 5 to 43 mass %, and further preferably 10 to 35 mass % with respect to the total mass of the needle-shaped portions 12. When it is 50 mass % or less, the shape of the needle-shaped portions 12 may readily be maintained, and the workability during production can be improved. When it is 3 mass % or more, it may be easier to increase the strength. When the filler is contained in this content range, it may be easy to increase the strength of the needle-shaped portions 12 by the filler while maintaining the liquid permeability by forming the needle-shaped portions 12 with a desired porosity. Two or more types of the above-described fillers may be contained. Also in this case, it may be preferred to contain the filler so that the total amount of the filler is within the above content range with respect to the resin constituting the needle-shaped portions 12.

The shape of the filler may be a plate-like (flake-like) shape, a fibrous shape, a spherical shape, an indefinite shape, or the like, but may be preferably a fibrous shape. When the shape of the filler is a fibrous shape, it is more compatible with the molten low-melting resin and the strength of the needle-shaped portions 12 obtained is more readily improved, which may be preferred. Examples of fillers having a fibrous shape include metal fiber filler, carbon fiber, carbon nanofiber, and cellulose fiber. When the filler is in a shape other than a fibrous shape, for example, when the filler is in a spherical shape or an indefinite shape, the filler may be composed of a resin whose glass-transition temperature is 80° C. or lower, as described above, thereby to allow the filler to be more compatible with the low-melting resin. The particle diameter of the filler may be 0.3 to 150 μm, preferably 0.5 to 125 μm, and more preferably 1 to 100 μm. When the particle diameter of the filler is 0.3 to 150 μm, the filler may be more readily dispersed in a composition containing a low-melting resin, and the strength of the microneedle structure 10 obtained can be further improved. The particle diameter of the filler is a seven-point average of the values obtained through observing the filler in the microneedle structure 10 with a scanning electron microscope (SEM) and measuring the lengths of the longest portions of the particles. When the filler is in a fibrous shape, the particle diameter refers to the fiber length.

As described above, in the first embodiment, the needle-shaped portions 12 are described as being composed of a high-melting-point resin, but the needle-shaped portions 12 may contain a resin other than the high-melting-point resin. In this case, the ratio of the high-melting-point resin to the total mass of the resin components contained in the needle-shaped portions 12 may be preferably 30 mass % or more, more preferably 50 mass % or more, and further preferably 70 mass % or more from the viewpoint of efficiently obtaining the effect of increasing the strength of the needle-shaped portions 12. Resins other than the high-melting-point resin contained in the needle-shaped portions 12 include low-melting-point resins whose melting point is lower than 130° C. Examples of the low-melting-point resins include polycaprolactone (melting point: 60° C.), polybutylene succinate (melting point: 84° C. to 115° C.), and aliphatic aromatic copolyester (melting point: 110° C. to 120° C.). The low-melting-point resin contained in the needle-shaped portions 12 together with the high-melting-point resin may be the same as the low-melting-point resin used in the above-described second embodiment, and may have a melting point of 130° C. or lower. By containing both the high-melting-point resin and the low-melting-point resin in the needle-shaped portions 12, it is possible to melt the resin at a low temperature while improving the tip strength of the needle-shaped portions 12. In this case, the high-melting-point resin and the low-melting-point resin may be in a kneaded state, but by using a high-melting-point resin as the resin used for the above-described filler and mixing the high-melting-point resin in the form of filler with the low-melting-point resin, it may be easier to mix the high-melting-point resin and the low-melting-point resin at a low temperature.

The needle-shaped portions 12 are each formed with the hole portions 13 as flow channels that allow liquids to flow inside. One or more hole portions 13 may be formed in one needle-shaped portion 12 and opened at the surface of the needle-shaped portion 12. In the present embodiment, the needle-shaped portions 12 may be formed with porous structures. When each needle-shaped portion 12 is formed so that at least a part thereof has a porous structure, body fluids or drug solutions can pass through the hole portions 13 of the porous structure, so this may be preferred because nano-order flow paths are not necessary to be mechanically formed. Moreover, body fluids or drug solutions can flow through all the flow paths of the portion formed with the porous structure in each needle-shaped portion 12, and the amount of flow can therefore be increased as compared with when a simple single communicating hole is formed. On the other hand, when each needle-shaped portion 12 is formed so that at least a part thereof has a porous structure, there is a possibility that the needle-shaped portions 12 may become brittle. For example, when the porous structure is not covered partially or entirely on the side surface of a needle-shaped portion 12, the hole portions 13 are also opened on the side surface of that needle-shaped portion 12. In this case, the amount of flow of the liquid can be increased as compared with when only the tip portion of a needle-shaped portion 12 is opened. When a needle-shaped portion 12 is formed with a porous structure or the hole portions 13 are opened on the side surface of that needle-shaped portion 12, it is conceivable that the needle-shaped portions 12 may become brittle. Fortunately, however, in the present embodiment, the needle-shaped portions 12 are formed having a tip strength within a predetermined value range, and it is therefore possible to form the needle-shaped portions 12 which are not brittle and are less likely to be defective or broken.

The method of forming the porous structures will be described in detail later, but a method of forming the porous structures simultaneously with the formation of the needle-shaped portions 12, or a method of forming projecting portions 32 formed with no porous structures (not illustrated in FIG. 1, described later) and then forming porous structures in the projecting portions 32, may be preferred from the viewpoint of obtaining the hole portions 13 with a continuous structure. In the latter case, for example, the porous structures may be obtained through mixing two or more different materials to form the projecting portions 32 and then removing at least one material to form the hole portions. When the needle-shaped portions 12 contain a filler, this method of forming porous structures may be used to contain the filler in a dispersed state in the resin of the needle-shaped portions 12. In the present embodiment, the needle-shaped portions 12 are formed in a production process described later that includes creating the projecting portions 32 composed of a water-insoluble high-melting-point resin and a water-soluble material, removing the water-soluble material, which is soluble in water, in a removal step to form the hole portions 13, and leaving the water-insoluble high-melting-point resin, which is insoluble in water, to form the porous needle-shaped portions 12.

In one aspect of the present embodiment, the hole portions 13 are voids formed by removing the water-soluble material from the projecting portions 32 composed of the water-insoluble high-melting-point or low-melting-point resin and the water-soluble material, and the body fluid or drug solution passes through the hole portions 13 which serve as flow channels. As illustrated in the cross sections of the needle-shaped portions 12, the hole portions 13 are formed by removing the water-soluble material to form a plurality of voids that communicate with each other. Some of the hole portions 13 may communicate from the surfaces of the needle-shaped portions 12 to one surface of the base material 11 to form the flow channels. The size of openings of the hole portions 13 is determined by the application such as a test patch using the microneedle structure 10, but from the viewpoint of facilitating the passage of liquids, the size of the openings may be preferably 0.1 to 50.0 μm, more preferably 0.5 to 25.0 μm, and further preferably 1.0 to 10.0 μm. In order to obtain such an opening diameter, the water-soluble material and its content may be appropriately selected in the production steps.

In one aspect of the present embodiment, the needle-shaped portions 12 are formed with porous structures by removing the water-soluble material from the projecting portions 32 composed of the water-insoluble high-melting-point or low-melting-point resin and the water-soluble material, but the method is not limited to this. It may also be possible to form the needle-shaped portions 12 using a porous high-melting-point resin, to form porous structures simultaneously with the formation of the needle-shaped portions 12 using a foaming material or the like, or to form porous structures by sintering a particulate composition containing a high-melting-point resin.

The needle-shaped portions 12 may be provided with a base portion 14 that is provided between the needle-shaped portions 12 and one surface side of the base material 11 over at least a region where the needle-shaped portions 12 are formed. In the present embodiment, the base portion 14 is provided in a layered form over the entire one surface of the base material 11. The base portion 14 serves as a base for each needle-shaped portion 12 and has hole portions 13 similarly to each needle-shaped portion 12. The base portion 14 may be formed to have a thickness, for example, of 0.1 to 500 μm. With such a thickness, the strength of the base material 11 is increased, and preferred adhesive properties are obtained between the needle-shaped portions 12, the base portion 14, and the base material 11.

Like the needle-shaped portions 12, the base portion 14 preferably has a porous structure, and it may be more preferred to use the same material with porous structure as that of the needle-shaped portions 12. When a porous structure is used for the base portion 14, there is no need to mechanically form hole portions 13 because flow channels through which liquids flow are formed inside the porous structure, and liquids from the needle-shaped portions 12 can pass through the hole portions 13 of the base portion 14 to fill the through-holes 15, which may be preferred. In the present embodiment, the base portion 14 is composed of the same high-melting-point resin as the material described for the needle-shaped portions 12 and is formed by the same steps; therefore, not only can the base portion 14 be easily created, but also better adhesion can be achieved between the needle-shaped portions 12 and the base material 11 via the base portion 14, which may be preferred. Furthermore, in the present embodiment, since the base portion 14 is provided over the entire one surface of the base material 11, the base portion 14 is present in a state of being attached to the base material 11 even in the portion of the base material 11, which is not formed with the needle-shaped portions 12, and the strength of the microneedle structure 10 is further improved as a whole.

### (2) Base Material

In the case of the above-described first embodiment, that is, when the needle-shaped portions 12 contain a high-melting-point resin, preferably, the base material 11 may have a layer containing a heat-resistant resin and may be configured such that a liquid can pass through the base material 11 in its thickness direction. The layer containing a heat-resistant resin may be sufficient if it contains the heat-resistant resin to an extent that the layer can exhibit heat resistance. The ratio of content of the heat-resistant resin to the total mass of the resin components contained in the layer containing the heat resistant resin may be preferably 50 mass % or more, more preferably 65 mass or more, and further preferably 80 mass % or more. Examples of the heat-resistant resin include heat-resistant organic polymers and silicone resins. The glass-transition temperature of the heat-resistant organic polymer may be preferably 80° C. or higher, more preferably 110° C. or higher, further preferably 140° C. or higher, and furthermore preferably 200° C. or higher. The glass-transition temperature of a heat-resistant organic polymer refers to a temperature determined through performing thermo-mechanical analysis (TMA) on a sample of the heat-resistant organic polymer at a heating rate of 5° C./min and calculating the temperature at the intersection of tangent lines before and after the inflection point of the obtained chart. By the base material 11 having a layer containing a heat-resistant resin, when the composition containing a high-melting-point resin is melted at a temperature equal to or higher than its melting point to bond the base material 11 upon production of the needle-shaped portions 12 in the production steps or when the composition containing a high-melting-point resin is melted at a temperature equal to or higher than its melting point to form the needle-shaped portions 12, even if the base material 11 is heated at the same time, it is possible to suppress deformation or deterioration of the base material 11. Since the melting point of the high-melting-point resin is 130° C. or higher, the possibility of obtaining such an effect can be further increased when the glass-transition temperature of the heat-resistant organic polymer is 140° C. or higher.

The feature that a liquid can pass through the base material 11 in its thickness direction preferably refers to a feature that the base material 11 is configured such that a liquid can pass through the base material 11 in its thickness direction via the through-holes 15 formed in the base material 11, while being configured of a material that is impermeable to liquids, rather than a feature that the base material 11 itself is configured of a material that is permeable to liquids. When the base material 11 has liquid impermeability, liquid absorption of the base material 11 can be suppressed, so the liquid can only pass through the through-holes 15 in the base material 11. Therefore, the body fluid obtained from the needle-shaped portions 12 or the drug solution transported to the needle-shaped portions 12 does not seep into the base material 11, so that the entire amount can be transported via the through-holes 15. Through this configuration, when the microneedle structure 10 is used as a test patch, a rapid analysis is possible because the body fluid can pass through the base material 11 in a moment, while also when the microneedle structure 10 is used as a drug administration patch, the drug solution does not seep and the total amount of the drug solution can be quickly supplied to the skin.

The layer of the base material 11 containing a heat-resistant resin may be more preferably formed of a flexible material that has high followability to the skin. The layer containing a heat-resistant resin may be preferably a resin film containing a heat-resistant resin from the viewpoint of imparting liquid impermeability to the base material 11. As described above, examples of the heat-resistant resin include heat-resistant organic polymers and silicone resins. The heat-resistant organic polymers may be preferably at least one selected from polymethylmethacrylate, polystyrene, polyacrylonitrile, polyphenylene oxide, polyethylene naphthalate, polyphenylene sulfide, polytetrafluoroethylene, polycarbonate, allyl resin, polyether ether ketone, acetyl cellulose resin, polysulfone, polyether sulfone, polyimide, and polyamide imide. Among the heat-resistant organic polymers, at one least selected from polyethylene naphthalate, polyphenylene sulfide, polytetrafluoroethylene, polycarbonate, allyl resin, polyether ether ketone, acetyl cellulose resin, polysulfone, polyether sulfone, polyimide, and polyamide imide may be more preferred, at least one selected from polycarbonate, allyl resin, polyether ether ketone, acetyl cellulose resin, polysulfone, polyether sulfone, polyimide, and polyamide imide may be further preferred, and at least one selected from polyether sulfone, polyimide, and polyamide imide may be furthermore preferred. As the heat-resistant organic polymers, copolymers obtained by copolymerizing the monomers that are the raw materials of these heat-resistant organic polymers and any other monomers may be used. Examples of such heat-resistant organic polymers include an acrylonitrile/butadiene/styrene copolymer. In general, the higher the glass-transition temperature of the above-described heat-resistant organic polymer, the higher the heat resistance of the heat-resistant organic polymer. For example, the general glass-transition temperature of polyimide is 300° C. or higher.

In the case of the above-described second embodiment, that is, when a low-melting-point resin whose melting point is 130° C. or lower is used as the resin forming the needle-shaped portions 12, the composition containing the low-melting-point resin can be processed at low temperatures, so that the base material 11 can be prevented from being exposed to high temperatures. Therefore, in place of a resin film containing a heat-resistant resin as the layer containing a heat-resistant resin, a resin film using a resin such as polybutylene terephthalate, polyethylene terephthalate, polyethylene, polypropylene, ethylene-vinyl acetate copolymer, vinyl chloride, acrylic resin, polyurethane, or polylactic acid may be adopted as the layer constituting the base material, and even with a resin film using such a resin, problems such as deformation of the base material are less likely to occur.

The base material 11 may be a single layer or may have a configuration in which multiple layers are laminated. The thickness of the base material 11 may be preferably 3 to 200 μm, more preferably 10 to 140 μm, and further preferably 30 to 115 μm. When the thickness is 3 μm or more, it is easy to maintain the strength as the base material 11, and when the thickness is 200 μm or less, the followability to the skin is improved and the liquid transport time can be shortened.

The base material 11 may also be provided with an adhesive layer 16. In the present embodiment, an adhesive layer 161 is provided on the surface of the base material 11 opposite to the surface (one surface) on which the needle-shaped portions 12 are formed. The provision of this adhesive layer 161 has an advantage that, when a tape is laminated on the surface of the base material 11 opposite to the one surface to cover an analysis sheet or the like, as will be described later, it is easy to adhere the tape to the base material 11, or the adhesiveness between the tape and the base material 11 is improved. As the adhesive constituting such an adhesive layer 161, a pressure sensitive adhesive may be preferred, and examples thereof include an acrylic-based pressure sensitive adhesive, a silicone-based pressure sensitive adhesive, and a rubber-based pressure sensitive adhesive, among which the acrylic-based pressure sensitive adhesive can be more preferably used.

Moreover, by providing an adhesive layer 162 on the surface of the base material 11 on which the needle-shaped portions 12 are formed, the microneedle structure 10 can be easily obtained in a method for producing a microneedle structure, which will be described later, through preliminarily making a solid composition 31 (not illustrated in FIG. 1, described later) adhere to the base material 11, putting the base material 11 and the solid composition 31 into a mold, and heating and pressing them in a formation step. When the adhesive layer 162 is provided on the base material 11, there is a concern that a gap may be generated between the base material 11 and the needle-shaped portions 12, causing liquid to leak out, or that the adhesive layer may prevent the passage of liquids between the base material 11 and the needle-shaped portions 12. For this reason, it may be preferred to provide the adhesive layer 162 so that it surrounds a region of the base material 11 through which the liquids should pass, while providing a central region in which the adhesive layer 162 is not formed. Although the microneedle structure 10 cannot be obtained in such a simple manner, a first primer layer (not illustrated) may be provided as substitute for the adhesive layer 162, for example, for the purpose of improving the adhesiveness between the needle-shaped portions 12 and the base material 11. Even when the base material 11 has the adhesive layer 162, the first primer layer as an intermediate layer may be provided between the base material 11 and the adhesive layer 162. Examples of the primer layer include an acrylic-based primer layer and a polyester-based primer layer.

As the acrylic-based pressure sensitive adhesive, one containing an acrylic polymer obtained by polymerizing a monomer whose main component is an alkyl acrylate can be used. The acrylic-based polymer may be a copolymer of an alkyl acrylate and another monomer. Examples of the other monomer include acrylic esters other than alkyl acrylates, such as acrylic esters having a hydroxyl group, acrylic esters having a carboxyl group, and acrylic esters having an ether group and monomers other than acrylic esters, such as vinyl acetate and styrene.

The acrylic-based polymer may be crosslinked by a reaction between a functional group derived from the above-described acrylic ester having a hydroxyl group, acrylic ester having a carboxyl group, etc., and a crosslinker.

The acrylic-based pressure sensitive adhesive may contain a tackifier, a plasticizer, an antistatic, a filler, a curable component, etc. in addition to the above-described components. As a coating liquid for obtaining the acrylic-based pressure sensitive adhesive, any of a solvent-based one and an emulsion-based one can be used.

The base material 11 is provided with a plurality of through-holes 15. In the present embodiment, since the needle-shaped portions 12 and the base portion 14 have porous structures, there is no need to align the through-holes 15 with the needle-shaped portions 12, and liquids can flow through the through-holes 15 by appropriately forming them. Thus, the microneedle structure 10 can be easily formed. In the present embodiment, the shape of the through-holes 15 provided in the base material 11 is circular when viewed from above, but is not limited to this and may be rectangular or the like. The through-holes 15 have an opening diameter to an extent that causes capillary action, and from the viewpoint of ensuring a sufficient amount of liquid flow, it may be preferred to provide a plurality of through-holes 15 in the base material 11. The diameter of the through-holes 15, when they are in a circular shape, for example, may be 2 mm or less, preferably 0.05 to 1 mm, and more preferably 0.1 to 0.8 mm. In the present embodiment, when transporting a liquid from the needle-shaped portions 12, the liquid does not seep into the base material 11 because the base material 11 has liquid impermeability, and the transportation distance is short because the liquid flows through the through-holes 15 in the thickness direction of the base material 11; therefore, when configured as a detection patch, it can perform the detection at a high analysis speed, while when configured as a drug administration patch, it can administer the drug solution early.

The sum of the areas of the through-holes 15 (total area) may be preferably 0.05% to 15%, more preferably 0.75% to 10%, and further preferably 1% to 5% in total with respect to the area of the region on the base material 11 in which the through-holes 15 are provided. When the total area of the through-holes 15 is 15% or less with respect to the area of the above region, the rigidity of the base material 11 can be readily ensured. On the other hand, when the total area of the through-holes 15 is 0.05% or more with respect to the area of the above region, the body fluid can be more efficiently acquired via the base material 11.

When the base portion 14 is formed in the microneedle structure 10, the base portion 14 is directly bonded to one surface of the base material 11, and the base portion 14 is integrally formed with the needle-shaped portions 12, so that the needle-shaped portions 12 are provided on the base material 11 without the use of adhesive or the like, and the hole portions 13 well communicate one another, allowing liquids to pass through easily. The microneedle structure 10 having such a configuration can be obtained, even when the adhesive layer 16 is not provided on the base material 11, through bonding the solid composition 31 to the base material 11 by heating in the formation step in the method for producing a microneedle structure, which will be described below, or through a similar bonding method using heating. In this case, it is necessary to heat the composition containing the high-melting-point resin to a temperature at which it can be bonded to the base material 11, but in the present invention, since the base material 11 has a layer composed of a heat-resistant resin, deformation or deterioration of the base material 11 due to heating can be suppressed. In the present embodiment, the base portion 14 is provided over the entire surface of the base material 11, but the present invention is not limited to this. The base portion 14 may be preferably formed at least in the region in which the needle-shaped portions 12 are formed. Even when the base portion 14 is directly bonded to one surface of the base material 11, as described above, the base material 11 may be provided with the first primer layer as substitute for the adhesive layer 16, and the base portion 14 may be bonded to the base material 11 via the first primer layer, or via a layer other than the adhesive layer 16 and the first primer layer.

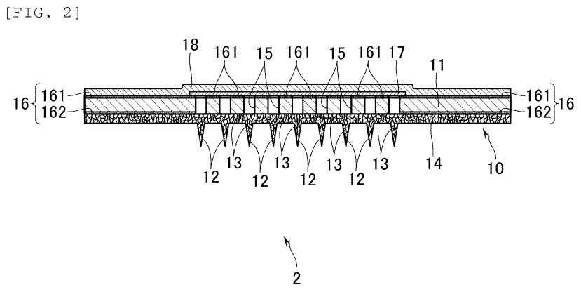

The microneedle structure 10 thus formed can be used as a test patch or a drug administration patch. For example, in a test patch 2 illustrated in FIG. 2, an analysis sheet 17 is disposed in a position covering the region of the base material 11 of the obtained microneedle structure 10 in which the through-holes 15 are formed, facing the needle-shaped portions 12, and a tape 18 is laminated to cover the analysis sheet 17. In the case of a drug administration patch, it may be configured such that a drug administration member is disposed as substitute for the analysis sheet 17 in a region position of the base material 11 of the obtained microneedle structure 10 in which the through-holes 15 are formed, facing the needle-shaped portions 12, and a tape 18 is laminated to cover the drug administration member. In such a test patch 2 or drug administration patch, since the strength of the needle-shaped portions 12 is high, it is possible to pierce the needle-shaped portions 12 into the skin without the defects, and the constituent material of the needle-shaped portions 12 can be prevented from remaining in the body, which may be preferred. The tape 18 for fixing the analysis sheet 17 or drug administration member to the base material 11 may be a pressure sensitive adhesive tape provided with a pressure sensitive adhesive layer.

### <Method for Producing Microneedle Structure>

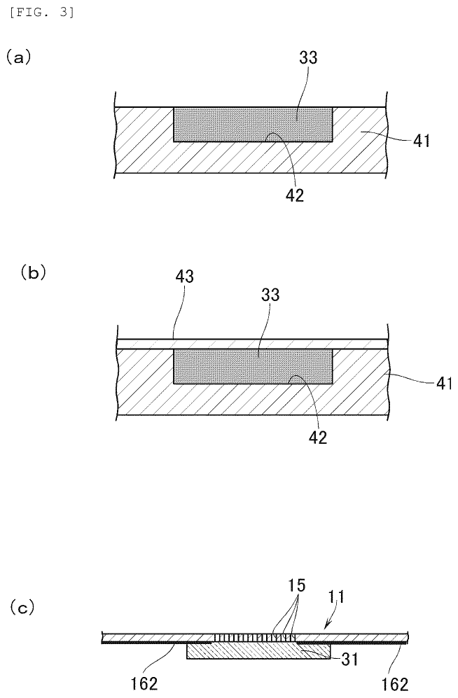

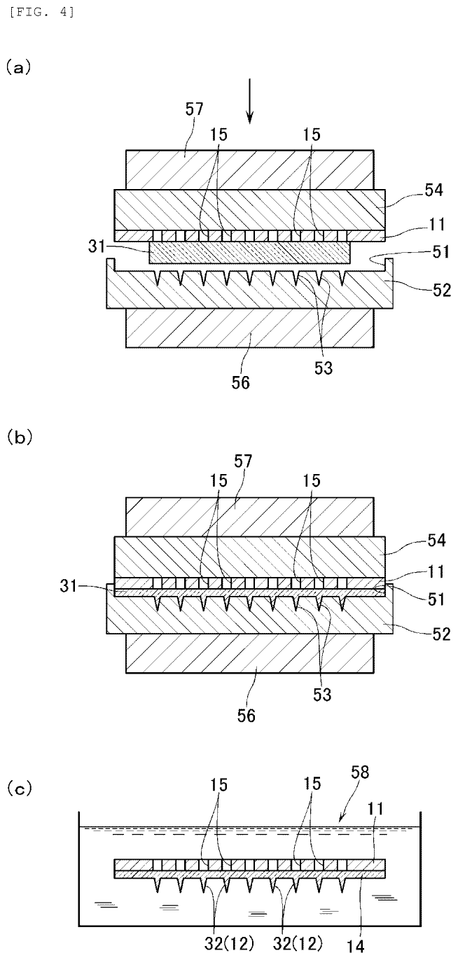

FIGS. 3 and 4 illustrate a method for producing the microneedle patch 1 according to an embodiment of the present invention. The method of the present embodiment includes bonding a mixture 33 containing a high-melting-point resin and a water-soluble material to the base material 11 to obtain the solid composition 31 with base material (bonding step), then heating and pressurizing the solid composition to form the projecting portions 32 (formation step), and thereafter removing the water-soluble material from the projecting portions 32 (removal step) to form the projecting portions 32 into the needle-shaped portions 12. This will be described in detail below.

### (Bonding Step)

Creation of the base material 11 and solid composition 31 will first be described. First, the high-melting-point resin and the water-soluble material are heated to melt and mixed to prepare the mixture 33, which is a composition containing the high-melting-point resin. In the present embodiment, the high-melting-point resin is water insoluble. In preparation of the mixture 33, in order that the viscosity is reduced when the resin is melted, heating may be preferably performed at a temperature that is equal to or higher than a temperature higher by 5° C. than the melting point of the high-melting-point resin and that is equal to or lower than a temperature higher by 80° C. than the melting point of the high-melting-point resin, more preferably at a temperature that is equal to or higher than a temperature higher by 10° C. than the melting point of the high-melting-point resin and that is equal to or lower than a temperature higher by 70° C. than the melting point of the high-melting-point resin, and further preferably at a temperature that is equal to or higher than a temperature higher by 15° C. than the melting point of the high-melting-point resin and that is equal to or lower than a temperature higher by 60° C. than the melting point of the high-melting-point resin. For example, when polylactic acid whose melting point is 170° C. is used as the high-melting-point resin, heating may be preferably performed at 175° C. to 250° C., more preferably at 180° C. to 240° C., and further preferably at 185° C. to 230° C. The mixture 33 may be preferably in a molten state. When it is important to perform heating at a lower temperature, the mixture 33 may be softened to an extent that allows it to be bonded to the base material 11, but in consideration of reducing the production time, etc., it may be preferred to heat the mixture 33 at a temperature equal to or higher than the melting point of the high-melting-point resin, as described above, at which the water-insoluble material begins to melt.

As the water-soluble material, a water-soluble material having at least a melting point higher than an ordinary temperature may be preferred. The water-soluble material may be organic or inorganic, and examples thereof include sodium chloride, potassium chloride, salt cake, sodium carbonate, potassium nitrate, alum, sugar, and water-soluble resin. The water-soluble resin may be preferably a water-soluble thermoplastic resin, and may preferably have a melting point higher than an ordinary temperature. Examples of the water-soluble thermoplastic resin include hydroxypropylcellulose and polyvinylpyrrolidone in addition to biodegradable resins, which will be described below. The water-soluble thermoplastic resin may be more preferably a biodegradable resin in consideration of the influence on the human body. Such biodegradable resins include at least one selected from the group consisting of polyalkylene glycols such polyethylene glycol and polypropylene glycol, polyvinyl alcohol, collagen, and a mixture thereof, and polyalkylene glycol may be particularly preferred. The molecular weight of polyalkylene glycol is, for example, preferably 200 to 4,000,000, more preferably 600 to 500,000, and particularly preferably 1,000 to 100,000. It may be preferred to use polyethylene glycol among polyalkylene glycols.

The water-soluble resin may be preferably a water-soluble resin whose melting point is 200° C. or lower, and the melting point may be more preferably 30° C. to 180° C. and further preferably 35° C. to 150° C. When the melting point is 150° C. or lower, an effect can be obtained that the water-soluble resin can be readily melted at the heating temperature for melting the high-melting-point resin. In order that both the high-melting-point resin and the water-soluble material can be readily melted at the same heating temperature when preparing the mixture 33, the difference between the melting point of the high-melting-point resin and the melting point of the water-soluble material may be preferably 40° C. or less and more preferably 30° C. or less.

The water-insoluble high-melting-point resin and the water-soluble material may be preferably mixed at a mass ratio of 9:1 to 1:9, more preferably 8:2 to 2:8, and particularly preferably 7:3 to 3:7. When the mixture 33 is configured in this ratio, the needle-shaped portions 12 having a desired porosity can be formed, and the needle-shaped portions 12 can readily achieve both the liquid permeability and the strength.

The mixture 33 may contain not only the water-insoluble material and the water-soluble material but also other materials as nonvolatile solids. For example, in order to further increase the strength of the needle-shaped portions, a water-insoluble material other than resin, such as silica filler, may be contained.

As illustrated in FIG. 3(a), the mixture 33 is injected into a recessed portion for solid composition 42 formed in a mold for solid composition 41 (filling step). The recessed portion for solid composition 42 may be formed with a shape and a capacity that allow a desired amount of the mixture 33 to be stored.

The material of the mold for solid composition 41 is also not particularly limited, but it is preferably formed, for example, of a silicone compound or the like, which facilitates the creation of an accurate mold and allows the solid composition 31 obtained by solidification to be readily released. In the present embodiment, the mold for solid composition 41 is composed of polydimethylsiloxane.

In a state in which the mixture 33 is stored in the recessed portion for solid composition 42, a sheet for solid composition 43 composed, for example, of polydimethylsiloxane (PDMS) is placed as a lid on the upper surface of the recessed portion for solid composition 42 to flatten the surface of the solid composition 31 obtained. By holding the entire mold for solid composition 41 at −10° C. to 3° C. for 1 to 60 minutes, the molten mixture 33 solidifies and becomes solid, so it is released with the sheet for solid composition 43 from the mold for solid composition 41, and the sheet for solid composition 43 is then removed. Through this operation, the solid composition 31 illustrated in FIG. 3(b) is obtained.

The base material 11 is also prepared. In the present embodiment, the base material 11 has an adhesive layer 162 on the surface of the base material 11 on which the needle-shaped portions 12 are formed. The adhesive layer 162 may be formed by coating or application, but in the present embodiment, a pressure sensitive adhesive tape having the adhesive layer 162 in a predetermined region is used as the base material 11. Then, through-holes 15 are formed in the base material 11. The method of forming the through-holes 15 is not particularly limited, and the through-holes 15 may be formed, for example, by laser perforation.

Then, as illustrated in FIG. 3(c), the solid composition 31 is attached to the adhesive layer 162 of the base material 11 to integrate the base material 11 and the solid composition 31. Thus, by having the adhesive layer 162, the microneedle structure 10 can be easily obtained through preliminarily making the solid composition 31 adhere to the base material 11 and placing the base material 11 and the solid composition 31 in a mold to heat and press them in the formation step, which will be described later. Moreover, the base material 11 and the solid composition 31 are integrated, so the handling such as transportation will be easier.

Then, as illustrated in FIG. 4(a), the solid composition 31 with the base material 11 is placed in a recessed portion 51 of a mold 52. Projection forming recessed portions 53 are also provided around the center of the bottom surface of the recessed portion 51. The solid composition 31 is placed on the projection forming recessed portions 53. The projection forming recessed portions 53 are for forming the needle-shaped portions 12 and are formed in a shape and size corresponding to the needle-shaped portions 12. Then, a lid 54 of the mold 52 is installed on the other surface side (back surface side) of the base material 11. This lid 54 is also composed, for example, of polydimethylsiloxane.

### (Formation Step)

Then, the formation step illustrated in FIG. 4(b) is performed. The formation step is for forming the projecting portions 32 having a desired form, etc., and the heating and pressurization may be performed once, but as in the present embodiment, in order to sufficiently fill the recessed portion 51 of the mold 52 with the solid composition 31, the formation step preferably includes a preliminary step for starting to melt the solid composition 31 provided with the base material 11 and a main step for sufficiently filling the recessed portion 51, etc. with the molten solid composition 31.

First, in the preliminary step and the main step, as illustrated in FIG. 4(b), the base material 11 and the solid composition 31 are interposed between the mold 52 and the lid 54 in a state in which the solid composition 31 is placed on the recessed portion 51. Then, in this state, the mold 52 and the lid 54 are placed on a lower stage 56, and an upper stage 57 is installed on the mold 52 and the lid 54.

As for the heating conditions in the preliminary step and the main step, the heating may be preferably performed at a temperature that is equal to or higher than a temperature higher by 10° C. than the melting point of the high-melting-point resin and that is equal to or lower than a temperature higher by 110° C. than the melting point of the high-melting-point resin, more preferably at a temperature that is equal to or higher than a temperature higher by 17° C. than the melting point of the high-melting-point resin and that is equal to or lower than a temperature higher by 95° C. than the melting point of the high-melting-point resin, and further preferably at a temperature that is equal to or higher than a temperature higher by 25° C. than the melting point of the high-melting-point resin and that is equal to or lower than a temperature higher by 80° C. than the melting point of the high-melting-point resin. For example, when polylactic acid whose melting point is 170° C. is used as the high-melting-point resin, the heating may be preferably performed at 180° C. to 280° C., more preferably at 187° C. to 265° C., and further preferably at 195° C. to 250° C. In the present embodiment, the heating is performed at a temperature at which the solid composition 31 can melt. In order to heat the solid composition 31, at least one of the lower stage 37 and the upper stage 57 may be heated or both may also be heated. In the main step, the heating may be maintained after the preliminary step, and the temperature may be changed as appropriate.

In this state, the mold 52 is pressed (pressurized) between the upper stage 57 and the lower stage 56. The pressure in this preliminary step is preferably 0.1 to 5.0 MPa. The pressure within this range allows the solid composition 31 to be melted in a short time and allows the molten solid composition 31 to quickly fill the recessed portion 51, etc. Then, the retention for 10 seconds to 10 minutes leads to a state in which the solid composition 31 is melted. The pressurization conditions may be changed between the preliminary step and the main step. For example, in the main step, pressurization can be performed at a higher pressure or for a longer time than in the preliminary step.

By performing the preliminary step and the main step as in the present embodiment, the solid composition 31 is sufficiently melted and fills the recessed portion 51 and the projection forming recessed portions 53. When the obtained needle-shaped portions 12 or base portion 14 has a porous structure, the bonding area of the needle-shaped portions 12 or the base portion 14 with respect to the base material 11 will be small, which may be disadvantageous for the bonding properties therebetween. Fortunately, however, the base material 11 and the solid composition 31 are subjected to the heating in the formation step in a state of being bonded to each other, and it can thereby be possible to improve the bonding properties between the needle-shaped portions 12 or the base portion 14 and the base material 11.

After that, the mold 52 is released from the lower stage 37, and the molten solid composition 31 is retained at −10° C. to 3° C. for 1 to 60 minutes to be refrigerated and solidified (refrigeration/solidification step). This allows the projecting portions 32, etc. to be formed, which have high transferability with a shape corresponding to the projection forming recessed portions 53.

### (Removal Step)

After the bonding step is completed, a product in which the solidified projecting portions 32 and the base material 11 are bonded to each other is released from the mold 52 and statically placed in a liquid, and a removal step is performed to remove the water-soluble material to form the needle-shaped portions 12.

The cleaning liquid in this removal step contains water, and in the present embodiment, as illustrated in FIG. 4(c), the removal step is performed by statically placing in a cleaning liquid 58 the product in which the projecting portions 32 and the base material 11 are bonded to each other. By statically placing it in the cleaning liquid containing water, portions exposed to outside or portions communicating with the portions exposed to outside in the water-soluble material contained in the projecting portions 32, etc. dissolve and flow into the water and are removed. The cleaning liquid is sufficient if it contains water, and may be, for example, a mixed solvent of water and alcohol, or the like. Through this removal, the hole portions 13 are formed in the projecting portions 32, etc., and the needle-shaped portions 12 composed of a water-insoluble component containing a high-melting-point resin is formed. Also in the molten solid composition 31 attached to one surface of the base material 11 by being filled in the recessed portion 51, the water-soluble material is removed so that the base portion 14 is also formed with the same porous structure as in the r needle-shaped portions 12. This allows the microneedle structure 10 of the present embodiment to be obtained. In the present embodiment, the microneedle structure 10 of the above-described first embodiment can be obtained.

### (Method for Producing Test Patch, Etc.)

Although not illustrated, a test patch can be produced through disposing the analysis sheet 17 at a predetermined position on the back surface side of the base material 11 of the obtained microneedle structure 10 and laminating the tape 18 so as to cover the analysis sheet 17 (installation step). A conventionally known method can be used as the lamination method. For example, a test patch can be produced through placing the analysis sheet 17 on the back surface side of the base material 11 and then laminating a pressure sensitive adhesive tape 18 in which a commonly-used adhesive layer of a rubber-based pressure sensitive adhesive, an acrylic-based pressure sensitive adhesive, a silicone-based pressure sensitive adhesive, or the like is laminated on a tape base material. A drug administration patch can also be produced by a similar method.

### Modification

In the present embodiment, the solid composition 31 has been described as containing the water-soluble material and the high-melting-point resin, but the solid composition 31 is not particularly limited, and may contain a low-melting-point resin or a filler. In this case, the microneedle structure 10 of the above-described second embodiment can be obtained. When the solid composition 31 is used as in the present embodiment, the composition does not contain a solvent, so discoloration and deformation of the base material 11 can be suppressed, which may be preferred. Furthermore, in the present embodiment, the bonding step may be carried out immediately after the filling step and before the mixture 33 is retained at a low temperature. That is, after filling the recessed portion 42 with the mixture 33, the base material and the mixture 33 may be brought into contact with each other, and the mixture 33 may be solidified to form the solid composition 31, thereby bonding the base material and the solid composition 31. In this case, the heat resistance of the base material 11 is exhibited even against the residual heat of the filling step.

In the present embodiment, the water-insoluble high-melting-point resin is used to form the needle-shaped portions 12 in order to readily form the hole portions 13 by removing the water-soluble material, but the method of creating the hole portions 13 is not particularly limited. For example, the formation step may include filling the mold 52 with a particulate high-melting-point resin or the like and sintering the high-melting-point resin at a temperature equal to or higher than the melting point of the high-melting-point resin thereby to obtain a microneedle structure having a porous structure composed of the sintered particles and a large number of voids formed between the particles. Also in this case, when the formation step and the bonding step are carried out at the same time, the base material 11 having a layer composed of a heat-resistant resin can suppress the deformation and deterioration of the base material 11.

In the present embodiment, the recessed portion 42 is filled with the mixture 33 to form the solid composition 31, but the present invention is not limited to this. For example, the formation step may adopt a scheme that includes: preparing a liquid composition to have a viscosity of 0.1 to 1000 mPa's in a state of containing a water-soluble material and a high-melting-point resin; and dropping the liquid composition with a dispenser or the like on the base material 11 thereby to form the projecting portions 32. Even when the solid composition 31 containing a high-melting-point resin is melted at a high temperature to form the projecting portions 32 and the base material is indirectly heated, the heat resistant base material 11 does not deform/soften, so the workability is good.

Furthermore, the bonding step may be performed after the formation step. In this case, upon the bonding between the projecting portions 32 or the like before the removal step or the needle-shaped portions 12 or the like after the removal step and the base material 11, even when the bonding involves heating, the heat-resistant base material 11 is not deformed/softened, and the workability is good.

The present invention will be described in more detail below with reference to Examples.

### EXAMPLES

### Example 1-1

Using a stirrer, 3 g of polyethylene glycol (PEG) (weight-average molecular weight 4,000, melting point 40° C.) as a water-soluble material and 7 g of polycaprolactone (PCL) (weight-average molecular weight 10,000) were stirred while being heated at 110° C. Furthermore, 0.5 g of ARBOCEL Natural Cellulose Fibers (average fiber length: 45 μm, available from Rettenmaier Japan Co., Ltd., containing 10 mass % of ignition residue at 850° C./4 hours other than cellulose fibers) was added and stirred again. In this way, the mixture 33 was prepared. The mold for solid composition 41 composed of polydimethylsiloxane was prepared, which was formed with the recessed portion 42 having a square opening of 15 mm×15 mm and a depth of 1.5 mm. The mixture 33 was injected into the recessed portion 42 of the mold for solid composition 41 so as to fill the recessed portion 42.

The sheet for solid composition composed of polydimethylsiloxane was placed as a lid on the mold for solid composition 41 into which the mixture 33 was injected, and the surface of the solid composition 31 was flattened. This state was maintained at 3° C. for 5 minutes, and the molten mixture 33 was solidified into a solid, so it was separated from the mold for solid composition 41 to obtain the solid composition 31. Then, the adhesive layer 16 of the base material 11, which was a pressure sensitive adhesive tape (a polyethylene terephthalate (PET) substrate (100 μm thick) formed with an acrylic-based pressure sensitive adhesive layer (25 μm thick) thereon), was attached to one surface of the solid composition 31 and was made to adhere to the solid composition 31. The solid composition 31 provided with the base material 11 was thus obtained.

To carry out the formation step, the mold 52 having the projection forming recessed portions 53 was prepared. The mold 52, composed of polydimethylsiloxane, was formed with the projection forming recessed portions 53 on its surface having the recessed portion 51 as detailed below:
Shape of the projection forming recessed portions: square pyramid shape with square cross section;
Length of one side of the maximum cross section of a projection forming recessed portion: 500 μm;
Height of the projection forming recessed portions: 900 μm;
Pitch of the projection forming recessed portions: 1,000 μm;
Number of the projection forming recessed portions: 13 in vertical row, 169 in total of 13 rows;
Size of the region formed with the projection forming recessed portions: 15 mm square; and
Arrangement of the projection forming recessed portions: square grid shape.

The preliminary step was carried out through: placing the mold 52 on the lower stage 56 of a heating press machine (available from AS ONE CORPORATION, AH-1T); placing the solid composition 31 with the base material 11 on the mold 52, facing the recessed portion 51; overlapping a sheet (lid 54) composed of polydimethylsiloxane and having a 30 mm square shape from above; and pressing them at 2 MPa for 1 minute 30 seconds while heating them at a lower stage setting heating temperature of 100° C. and an upper stage setting heating temperature of 90° C. of the heating press machine. After that, the main step was carried out by pressing at 4 MPa for 30 seconds in a heating state in which the temperatures of the heating press machine were retained. Furthermore, the base material 11 and the molten solid composition housed in the lid 54 and mold 52 were stored in a refrigerator at 3° C. for 5 minutes to solidify the solid composition, forming the projecting portions 32, etc. Thereafter, the base material 11 was released from the mold 52, and the base material 11 and the formed projecting portions 32, etc. were immersed in purified water at 23° C. for 24 hours to dissolve and remove the water-soluble material. After that, the base material 11 and the molded solid composition 31 were statically placed in a drying oven (30° C.) for 5 hours to evaporate water and dry, thus obtaining the microneedle structure 10.

### Example 1-2

The microneedle structure 10 was obtained in the same manner as in Example 1-1 except that 2.0 g of ARBOCEL Natural Cellulose Fibers was added as a filler.

### Example 1-3

The microneedle structure 10 was obtained in the same manner as in Example 1-1 except that 0.5 g of ARBOCEL Ultrafine Cellulose (average particle size: 6 to 12 μm, available from Rettenmaier Japan Co., Ltd.) was added as a filler in place of ARBOCEL Natural Cellulose Fibers.

### Example 1-4

The microneedle structure 10 was obtained in the same manner as in Example 1-1 except that 2.0 g of ARBOCEL Ultrafine Cellulose (average particle size: 6 to 12 μm, available from Rettenmaier Japan Co., Ltd.) was added as a filler in place of ARBOCEL Natural Cellulose Fibers.

### Example 1-5

The microneedle structure 10 was obtained in the same manner as in Example 1-1 except that 0.5 g of lactic acid polymer (PLA) particles (glass-transition temperature: 60° C., indefinite shape, average particle diameter: 53.4 μm) was added as a filler in place of ARBOCEL Natural Cellulose Fibers.

### Example 1-6

The microneedle structure 10 was obtained in the same manner as in Example 1-1 except that 0.5 g of TECHPOLYMER SSX (available from Sekisui Kasei Co., Ltd., true spherical crosslinked polymethyl methacrylate (PMMA) fine particles, glass-transition temperature of PMMA: 100° C., particle diameter: 1.5 μm) was added as a filler in place of ARBOCEL Natural Cellulose Fibers.

### Comparative Example 1-1

A microneedle structure was obtained in the same manner as in Example 1-1 except that ARBOCEL Natural Cellulose Fibers were not added as a filler.

The microneedle structures obtained in Examples 1-1 to 1-6 and Comparative Example 1-1 were subjected to the following microneedle array transferability evaluation and microneedle tip strength evaluation.

### (Microneedle Array Transferability Evaluation)

In each of the examples and comparative example, projecting portions were formed by cooling the composition, and after release from the mold and before immersion in purified water, the projecting portions were observed with an optical microscope (magnification: 50× and 100×) to count the number of projecting portions remaining on the base material. The ratio of this remaining number to the total number of projecting portions in the design was calculated to determine the transfer ratio. A transfer ratio of 50% or more and 100% or less was evaluated as “A,” and a transfer ratio of 0% or more and less than 50% was evaluated as “B.”

### (Microneedle Tip Strength Evaluation)

The microneedle structure obtained in each of the examples and comparative example was placed on a stage with the needle-shaped portions facing up, observation with a microscope was perform to select one needle-shaped body having a sharp tip shape, and an attachment (made of iron, 2 mmφ) of a measurement device (digital force gauge available from IMADA CO., LTD.) was aligned with the position of the needle-shaped portion of the needle-shaped body and was brought close to the needle-shaped portion, taking care not to allow the attachment to come into contact with adjacent needle-shaped portions. The attachment was moved up and down to a position at which it came into contact with the tip of the needle-shaped portion but no force was applied to the needle-shaped portion, and then the attachment was raised 0.1 mm from there and was thereafter lowered at a speed of 5 mm/min to start measurement of the force applied to the attachment (measurement range: 1 to 5,000 mN). At that time, the measurement temperature was 23° C. and the relative humidity was 50%. At a point of time when a decrease in the force was first observed on a graph on which the measured force was output, a local maximum value of the force exhibited at a position before the decrease in the force was read, or a value of the force at a point of time when the fall of the attachment reached 100 μm was read if the decrease in the force was not observed until the fall of the attachment reached 100 μm. The read value was determined as the tip strength of the needle-shaped portion. When the tip strength was 60 mN or more, it was evaluated as “A,” when the tip strength was less than 60 mN and 40 mN or more, it was evaluated as “B,” and when the tip strength was less than 40 mN, it was evaluated as “C.” For the examples or comparative example in which the transfer ratio was less than 100% in the transferability evaluation, one of the needle-shaped portions that remained on the base material without falling off was selected and evaluated. In all of Examples 1-1 to 1-6 and Comparative Example 1-1, a decrease in the force was observed before the fall of the attachment reached 100 μm.

The evaluation results are listed in Table 1.

TABLE 1

| | Resin constituting | | | Amount | | | | |
|---|---|---|---|---|---|---|---|---|
| | needle-shaped | Water-soluble | | of addition | | | | |
| | portions | material | | (to 100 parts | | Evaluation of | Tip strength | |
| | (PCL) (g) | (PEG) (g) | Filler | of resin) | | transferability | (mN/needle) | |
| Example 1-1 | 7 | 3 | CNF natural | 5 | parts | A | 99.75 | A |
| | | | cellulose fiber | | | | | |
| Example 1-2 | 7 | 3 | CNF natural | 20 | parts | A | 106 | A |
| | | | cellulose fiber | | | | | |
| Example 1-3 | 7 | 3 | CNF Ultrafine | 5 | parts | A | 103.16 | A |
| | | | cellulose | | | | | |
| Example 1-4 | 7 | 3 | CNF Ultrafine | 20 | parts | A | 118.7 | A |
| | | | cellulose | | | | | |
| Example 1-5 | 7 | 3 | PLA particles | 5 | parts | A | 120.6 | A |
| Example 1-6 | 7 | 3 | PMMA fine | 5 | parts | B | 52.6 | B |
| | | | particles | | | | | |

| | | | | | | | |
|---|---|---|---|---|---|---|---|
| Comparative | 7 | 3 | — | — | A | 36.8 | C |
| Example 1-1 | | | | | | | |

As listed in Table 1, the microneedle tip strength evaluation was C in Comparative Example 1-1 in which the microneedle tip strength was less than 40 mN, but in Examples 1-1 to 1-6, it was A or B, and the strength was further increased by including filler. In Examples 1-1 to 1-6, the microneedle array transferability evaluation was A or B, but in Examples 1-1 to 1-5, the adhesion at the interface between the resin constituting the needle-shaped portions 12 and the filler was high, and the resin and the filler were well compatible, so the transferability was high.

In Examples 2-1 and 2-2 and Comparative Example 2-1, the weight-average molecular weight (Mw) refers to an equivalent weight-average molecular weight measured with a standard substance: polystyrene standard under the following conditions using gel permeation chromatography (GPC) (GPC measurement). Samples for GPC measurement were prepared as follows. First, 1 g of polycaprolactone (PCL) used in the examples and comparative example and 9 g of tetrahydrofuran (THF, available from FUJIFILM Wako Pure Chemical Corporation) were added to a screw tube, shaken, and completely dissolved to prepare a 10% PCL solution. Furthermore, 1 ml of the obtained solution and 9 ml of THE were dropped into a separately prepared screw tube to prepare a 1% PCL solution. This 1% PCL solution was filtered with a GD/X syringe filter (available from Whatman) and dropped into a GPC device.

### (Measurement Conditions)

Measurement device: HLC-8320 available from Tosoh Corporation
GPC columns (passing through in the following order): available from Tosoh Corporation
   TSK gel super H-H
   TSK gel super HM-H
   TSK gel super H2000
Solvent for measurement: tetrahydrofuran
Measurement temperature: 40° C.

### Example 2-1

Using Labo-plastomill 4C150 (Toyo Seiki Seisaku-sho, Ltd.), 3 g of polyethylene glycol (PEG) (weight-average molecular weight: 4,000) as a water-soluble material and 7 g of pellet-like polycaprolactone (weight-average molecular weight 80,000) were heated and kneaded at 170° C. In this way, the mixture 33 was prepared. The mold for solid composition 41 composed of polydimethylsiloxane was prepared, which was formed with the recessed portion 42 having a square opening of 15 mm×15 mm and a depth of 1.5 mm. The mixture 33 was injected into the recessed portion 42 of the mold for solid composition 41 so as to fill the recessed portion 42.

The mold lid for solid composition (sheet composed of polydimethylsiloxane) 43 was placed on the mold for solid composition 41, and the surface of the solid composition 31 was flattened. This state was maintained at 3° C. for 5 minutes, and the molten mixture 33 was solidified into a solid, so it was separated from the mold for solid composition 41 to obtain the solid composition 31. Then, the pressure sensitive adhesive layer of the base material 11, which was a pressure sensitive adhesive tape (PET substrate (100 μm thick) formed with an acrylic-based pressure sensitive adhesive layer (25 μm thick) thereon), and the solid composition 31, were bonded to each other. The solid composition 31 provided with the base material 11 was thus obtained.

To carry out the formation step, the mold 52 having the projection forming recessed portions 53 was prepared. The mold 52, composed of polydimethylsiloxane, was formed with the projection forming recessed portions 53 on its surface having the recessed portion 51 as detailed below:
Shape of the projection forming recessed portions: square pyramid shape with square cross section;
Length of one side of the maximum cross section of a projection forming recessed portion: 500 μm;
Height of the projection forming recessed portions: 900 μm;
Pitch of the projection forming recessed portions: 1,000 μm;
Number of the projection forming recessed portions: 13 in vertical row, 169 in total of 13 rows;
Size of the region formed with the projection forming recessed portions: 15 mm square; and
Arrangement of the projection forming recessed portions: square grid shape.

The preliminary step was carried out through: placing the mold 52 on the lower stage 56 of a heating press machine (available from AS ONE CORPORATION, AH-1T); placing the solid composition 31 with the base material 11 on the mold 52, facing the recessed portion 51; overlapping a sheet (lid 54) composed of polydimethylsiloxane and having a 30 mm square shape from above; and pressing them at 2 MPa for 3 minutes while heating them at a lower stage setting heating temperature of 140° C. and an upper stage setting heating temperature of 140° C. of the heating press machine. After that, the main step was carried out by pressing at 4 MPa for 30 seconds in a heating state in which the temperatures of the heating press machine were retained. Furthermore, the base material 11 and the molten solid composition housed in the lid 54 and mold 52 were stored in a refrigerator at 3° C. for 5 minutes to solidify the solid composition, forming the projecting portions 32, etc. Thereafter, the base material 11 was released from the mold 52, and the base material 11 and the formed projecting portions 32, etc. were immersed in purified water at 23° C. for 24 hours to dissolve and remove the water-soluble material. After that, the base material 11 and the molded solid composition 31 were statically placed in a drying oven (30° C.) for 5 hours to evaporate water and dry, thus obtaining the microneedle structure 10.

### Example 2-2

The microneedle structure 10 was obtained in the same manner as in Example 2-1 except that 7 g of pellet-like polycaprolactone with a different molecular weight from that in Example 2-1 (weight-average molecular weight 40,000) was used as the resin constituting the needle-shaped portions 12 and the temperatures of the heating step in the formation step were all 110° C.

### Comparative Example 2-1

A microneedle structure was obtained in the same manner as in Example 2-1 except that 7 g of pellet-like polycaprolactone with a different molecular weight from that in Example 2-1 (weight-average molecular weight 10,000) was used as the resin constituting the needle-shaped portions 12, the temperatures of the heating step in the formation step were all 110° C., and the pressurization time in the preliminary step was 1 minute 30 seconds.

The microneedle structures obtained in Examples 2-1 and 2-2 and Comparative Example 2-1 were subjected to the following microneedle array transferability evaluation and microneedle tip strength evaluation.

### (Microneedle Array Transferability Evaluation)

In each of the examples and comparative example, projecting portions were formed by cooling the composition, and after release from the mold and before immersion in purified water, the projecting portions were observed with an optical microscope (magnification: 50× and 100×) to count the number of projecting portions remaining on the base material. The ratio of this remaining number to the total number of projecting portions in the design was calculated to determine the transfer ratio. A transfer ratio of 50% or more and 100% or less was evaluated as “A,” and a transfer ratio of 0% or more and less than 50% was evaluated as “B.”

### (Microneedle Tip Strength Evaluation)

The microneedle structure obtained in each of the examples and comparative example was placed on a stage with the needle-shaped portions facing up, observation with a microscope was perform to select one needle-shaped body having a sharp tip shape, and an attachment (made of iron, 2 mmφ) of a measurement device (digital force gauge available from IMADA CO., LTD.) was aligned with the position of the needle-shaped portion of the needle-shaped body and was brought close to the needle-shaped portion, taking care not to allow the attachment to come into contact with adjacent needle-shaped portions. The attachment was moved up and down to a position at which it came into contact with the tip of the needle-shaped portion but no force was applied to the needle-shaped portion, and then the attachment was raised 0.1 mm from there and was thereafter lowered at a speed of 5 mm/min to start measurement of the force applied to the attachment (measurement range: 1 to 5000 mN). At that time, the measurement temperature was 23° C. and the relative humidity was 50%. At a point of time when a decrease in the force was first observed on a graph on which the measured force was output, a local maximum value of the force exhibited at a position before the decrease in the force was read, or a value of the force at a point of time when the fall of the attachment reached 100 μm was read if the decrease in the force was not observed until the fall of the attachment reached 100 μm. The read value was determined as the tip strength of the needle-shaped portion. When the tip strength was more than 200 mN, it was evaluated as “A,” when the tip strength was 100 to 200 mN, it was evaluated as “B,” and when the tip strength was less than 100 mN, it was evaluated as “C.” For the examples or comparative example in which the transfer ratio was less than 100% in the transferability evaluation, one of the needle-shaped portions that remained on the base material without falling off was selected and evaluated. In Examples 2-1 and 2-2, a decrease in the force was not observed until the fall of the attachment reached 100 μm. On the other hand, in Comparative Example 2-1, a decrease in the force was observed before the fall of the attachment reached 100 μm.

The evaluation results are listed in Table 2.

TABLE 2

| | | | | | | | | Evaluation | | |
|---|---|---|---|---|---|---|---|---|---|---|
| | First water- | | | First water- | | Heating press | | | MN tip strength | |
| | insoluble material | | | soluble material | | temperature | | MN | | Measured |
| | | Molecular | | | Molecular | Preliminary | Main | transfer- | Determi- | value |
| | Material | weight | Shape | Material | weight | step | step | ability | nation | (mN) |
| Example 2-1 | PCL | 80,000 | Pellet | PEG | 4,000 | 140 | 140 | A | A | 282 |
| Example 2-2 | PCL | 40,000 | Pellet | PEG | 4,000 | 110 | 110 | A | B | 165 |
| Comparative | PCL | 10,000 | Pellet | PEG | 4,000 | 110 | 110 | A | C | 37 |
| Example 2-3 | | | | | | | | | | |

As listed in Table 2, in Examples 2-1 and 2-2 and Comparative Example 2-1, the microneedle tip strength evaluation was A. On the other hand, as listed in Table 2, the microneedle tip strength evaluation was C in Comparative Example 2-1 in which the microneedle tip strength was less than 40 mN. From this, it has been found that the strength of the needle-like portions 12 is maximized when a low-melting-point resin with a high molecular weight (weight-average molecular weight of 40,000 or more) is used.

### Example 3-1

A mixture was prepared through stirring with a stirrer 100 mass parts of polyethylene glycol (molecular weight 4,000, melting point 40° C.) as a water-soluble material and 100 mass parts of polylactic acid (melting point 170° C.) as a high-melting point material while heating them to 190° C., and melting and mixing them. The mold for solid composition 41 composed of polydimethylsiloxane was prepared, which was formed with the recessed portion 42 having a square opening of 15 mm×15 mm and a depth of 1.5 mm. The mixture 33 was injected into the recessed portion 42 of the mold for solid composition 41 so as to fill the recessed portion 42.

Then, the sheet for solid composition 43 composed of polydimethylsiloxane was placed as a lid on the mold for solid composition 41 into which the mixture 33 was injected, and the surface of the solid composition 31 was flattened. This state was maintained at 3° C. for 5 minutes, and the molten mixture 33 was solidified into a solid, so it was separated from the mold for solid composition 41 to obtain the solid composition 31.

Then, the adhesive layer 162 of the base material 11, which was a pressure sensitive adhesive sheet obtained by providing the adhesive layer 162 composed of an acrylic-based pressure sensitive adhesive with a thickness of 25 μm on a polyimide film (PI, available from DU PONT-TORAY CO., LTD., product name: Kapton, thickness: 25 μm), was attached to one surface of the solid composition 31 and was made to adhere to the solid composition 31. The solid composition 31 provided with the base material 11 was thus obtained.

To carry out the formation step, the mold 52 having the projection forming recessed portions 53 was prepared. The mold 52, composed of polydimethylsiloxane, was formed with the projection forming recessed portions 53 on its surface having the recessed portion 51 as detailed below:
Shape of the projection forming recessed portions: square pyramid shape with square cross section;
Length of one side of the maximum cross section of a projection forming recessed portion: 500 μm;
Height of the projection forming recessed portions: 900 μm;
Pitch of the projection forming recessed portions: 1,000 μm;
Number of the projection forming recessed portions: 13 in vertical row, 169 in total of 13 rows;
Size of the region formed with the projection forming recessed portions: 15 mm square; and
Arrangement of the projection forming recessed portions: square grid shape.

The preliminary step was carried out through: placing the mold 52 on the lower stage 56 of a heating press machine (available from AS ONE CORPORATION, AH-1T); placing the solid composition 31 with the base material on the mold 52, facing the recessed portion 51; overlapping a sheet (lid 54) composed of polydimethylsiloxane and having a 30 mm square shape from above; and pressing them at 2 MPa for 3 minutes while heating them only with the lower stage at a setting heating temperature of 230° C. of the heating press machine. After that, the main step was carried out in the same manner by pressing them at 4 MPa for 30 seconds while heating them only with the lower stage at 230° C. Furthermore, the base material 11 and the molten solid composition housed in the lid 54 and mold 52 were stored in a refrigerator at 3° C. for minutes to solidify the solid composition, forming the projecting portions 32, etc. Thereafter, the base material 11 was released from the mold 52, and the base material 11 and the composition were immersed in purified water at 23° C. for 24 hours to dissolve and remove the water-soluble material. After that, the base material 11 and the molded solid composition 31 were statically placed under an environment of 23° C. and relative humidity 50% for 24 hours to evaporate water and dry, thus obtaining the microneedle structure 10.

### Reference Example 3-1

A microneedle structure was created in the same manner as in Example 3-1 except that a pressure sensitive adhesive sheet in which an adhesive layer composed of an acrylic-based pressure sensitive adhesive with a thickness of 25 μm was provided on a polyethylene terephthalate (PET) film was used in place of the pressure sensitive adhesive sheet of Example 1.

### Comparative Example 3-1

In this comparative example, the needle-shaped portions were formed using a low-melting-point resin, polycaprolactone (melting point 60° C.), in place of the high-melting-point resin, polylactic acid, used in Example 3-1, and a pressure sensitive adhesive sheet in which an adhesive layer composed of an acrylic-based pressure sensitive adhesive with a thickness of 25 μm was provided on a polyethylene terephthalate (PET) film was used in place of the pressure sensitive adhesive sheet of Example 3-1. A microneedle structure was created in the same manner as in Example 3-1 except that the heating temperatures in the bonding step and formation step were as follows:
Heating temperature during preparation of the mixture in the bonding step: 100° C.;
Lower stage temperature, pressure, and time in the preliminary step in the formation step: 110° C., 2 MPa, and 3 minutes; and
Lower stage temperature, pressure, and time in the main step in the formation step: 110° C., 4 MPa, and 30 seconds.

The microneedle structures obtained in Example 3-1 and Comparative Examples 3-1 and 3-2 were subjected to the following microneedle array transferability evaluation, microneedle tip strength evaluation, and base material deformation evaluation.

### (Microneedle Array Transferability Evaluation)

In each of the example and comparative examples, projecting portions were formed by cooling the composition, and after release from the mold and before immersion in purified water, the projecting portions were observed with an optical microscope (magnification: 50× and 100×) to count the number of projecting portions remaining on the base material. The ratio of this remaining number to the total number of projecting portions in the design was calculated to determine the transfer ratio. A transfer ratio of 50% or more and 100% or less was evaluated as “A,” and a transfer ratio of 0% or more and less than 50% was evaluated as “B.”

### (Microneedle Tip Strength Evaluation)

The microneedle structure obtained in each of the example and comparative examples was placed on a stage with the needle-shaped portions facing up, observation with a microscope was perform to select one needle-shaped body having a sharp tip shape, and an attachment (made of iron, 2 mmφ) of a measurement device (digital force gauge available from IMADA CO., LTD.) was aligned with the position of the needle-shaped portion of the needle-shaped body and was brought close to the needle-shaped portion, taking care not to allow the attachment to come into contact with adjacent needle-shaped portions. The attachment was moved up and down to a position at which it came into contact with the tip of the needle-shaped portion but no force was applied to the needle-shaped portion, and then the attachment was raised 0.1 mm from there and was thereafter lowered at a speed of 5 mm/min to start measurement of the force applied to the attachment (measurement range: 1 to 5000 mN). At that time, the measurement temperature was 23° C. and the relative humidity was 50%. At a point of time when a decrease in the force was first observed on a graph on which the measured force was output, a local maximum value of the force exhibited at a position before the decrease in the force was read, or a value of the force at a point of time when the fall of the attachment reached 100 μm was read if the decrease in the force was not observed until the fall of the attachment reached 100 μm. The read value was determined as the tip strength of the needle-shaped portion. When the tip strength was 40 mN or more, it was evaluated as “A,” and when the tip strength was less than 40 mN, it was evaluated as “B.” For the example or comparative examples in which the transfer ratio was less than 100% in the transferability evaluation, one of the needle-shaped portions that remained on the base material without falling off was selected and evaluated.

### (Base Material Deformation Evaluation)

The shape of the base material of the obtained microneedle structure was visually observed. When there was not shrinkage, melting, bending, or discoloration of the base material, it was evaluated as “A,” and when there was at least one of shrinkage, melting, bending, or discoloration of the base material, it was evaluated as “B.”

The evaluation results are listed in Table 3.

TABLE 3

| | | | | Temperature of | | Evaluation | | |
|---|---|---|---|---|---|---|---|---|
| | High-melting-point resin | | | heating press | | | | Thermal |
| | | Melting | Type | Preliminary | Main | Microneedle | | deformation |
| | | point | of base | step | step | transferability | Microneedle | of base |
| | Type | (° C.) | material | (° C.) | (° C.) | evaluation | tip strength | material |
| Example 3-1 | Polylactic acid | 170 | Polyimide | 230 | 230 | A | A | A |
| Reference | Polylactic acid | 170 | PET | 230 | 230 | B | A | B |
| Example 3-1 | | | | | | | | |
| Comparative | Polycaprolactone | 60 | PET | 110 | 110 | A | B | A |
| Example 3-1 | | | | | | | | |

As listed in Table 3, in Example 3-1, the tip strength of the needle-shaped portions was 40 mN or more and the base material deformation evaluation was A, whereas in Reference Example 3-1, the base material deformation evaluation was B, and the microneedle structure of Comparative Example 3-1 was not able to achieve a tip strength of the needle-shaped portions of 40 mN or more.

### INDUSTRIAL APPLICABILITY

The microneedle structure of the present invention can be used as a test patch, for example, by placing an analysis sheet on the back surface side and laminating it with a tape.

### DESCRIPTION OF REFERENCE NUMERALS

10 Microneedle structure
11 Base material
12 Needle-shaped portion
13 Hole portion
14 Base portion
15 Through-hole
16 Adhesive layer
31 Solid composition
32 Projecting portion
33 Mixture

1. A microneedle structure comprising: a needle-shaped portion having an interior formed with a hole portion; and a base material having one surface side on which the needle-shaped portion is formed,
the needle-shaped portion being formed with a porous structure,
the needle-shaped portion having a value of tip strength of 40 mN or more as measured by an evaluation method below,
the evaluation method comprising:
placing the microneedle structure on a stage with the needle-shaped portion facing up;
observing the microneedle structure with a microscope to select one needle-shaped body having a sharp tip shape;
aligning an attachment (made of iron, 2 mmφ) of a digital force gauge with a position of the needle-shaped portion of the needle-shaped body;
lowering the attachment at a speed of 5 mm/min to measure a force applied to the attachment (measurement environment temperature: 23° C., measurement environment relative humidity: 50%);
at a point of time when a decrease in the force is first observed on a graph on which the measured force is output, reading a local maximum value of the force exhibited at a position before the decrease in the force, or reading a value of the force at a point of time when a fall of the attachment reaches 100 μm if the decrease in the force is not observed until the fall of the attachment reaches 100 μm; and
determining the read value as the tip strength of the needle-shaped portion.

2. The microneedle structure according to claim 1, wherein the hole portion is opened on a side surface of the needle-shaped portion.

3. The microneedle structure according to claim 1, wherein
the needle-shaped portion contains a high-melting-point resin whose melting point exceeds 130° C.,
the base material contains a layer containing a heat-resistant resin, and a liquid can pass through the base material in its thickness direction.

4. The microneedle structure according to claim 3, wherein the high-melting-point resin is a water-insoluble resin.

5. The microneedle structure according to claim 3, wherein the high-melting-point resin is a biodegradable resin.

6. The microneedle structure according to claim 3, wherein the high-melting-point resin is a copolymer of at least one monomer selected from polylactic acid and polyglycolic acid and another monomer.

7. The microneedle structure according to claim 3, wherein the layer containing a heat-resistant resin contains at least one heat-resistant organic polymer selected from polymethylmethacrylate, polystyrene, polyacrylonitrile, polyphenylene oxide, polyethylene naphthalate, polyphenylene sulfide, polytetrafluoroethylene, polycarbonate, allyl resin, polyether ether ketone, acetyl cellulose resin, polysulfone, polyether sulfone, polyimide, and polyamide imide, a copolymer obtained by copolymerizing a monomer that is a raw material of the heat-resistant organic polymer and any other monomer, or a silicone resin.

8. The microneedle structure according to claim 3, wherein the base material and the needle-shaped portion are directly bonded via a base portion formed of a same material as that of the needle-shaped portion.

9. The microneedle structure according to claim 1, wherein the needle-shaped portion contains a low-melting-point resin whose melting point is 130° C. or lower.

10. The microneedle structure according to claim 1, wherein the needle-shaped portion contains a high-melting-point resin and a low-melting-point resin whose melting point is 130° C. or lower.

11. A method for producing a microneedle structure comprising: a needle-shaped portion having an interior formed with a hole portion; and a base material having one surface side on which the needle-shaped portion is formed,
the method comprising a formation step of heating a composition containing a high-melting-point resin whose melting point exceeds 130° C. to form a projecting portion on the base material by the composition.

12. The method for producing a microneedle structure according to claim 11, wherein
the high-melting-point resin is a water-insoluble resin,
the composition is a mixture of the water-insoluble resin and a water-soluble material, and
the method comprises a removal step of, after the formation step, removing with a water-containing solution the water-soluble material of the formed projecting portion to form a hole portion in the projecting portion.

13. A method for producing a microneedle structure comprising: a needle-shaped portion having an interior formed with a hole portion; and a base material having one surface side on which the needle-shaped portion is formed,
the method comprising a bonding step of heating a composition containing a high-melting-point resin whose melting point exceeds 130° C. to bond the heated composition and the base material.
